(19) United States
(12) Patent Application Publication
(10) Pub. No.: US 2024/0392075 A1
(43) Pub. Date: Nov. 28, 2024

(54) SOLUTION PHASE POLYMER SYNTHESIS

(71) Applicant: EXACTMER LIMITED, Harrogate (GB)

(72) Inventors: Piers Robert James Gaffney, Harrogate (GB); Andrew Guy Livingston, Harrogate (GB)

(21) Appl. No.: 18/695,844

(22) PCT Filed: Oct. 14, 2022
(86) PCT No.: PCT/GB2022/052627

§ 371 Date: Mar. 27, 2024

(30) Foreign Application Priority Data
Oct. 14, 2021 (GB) 2114688.1

(51) Int. Cl. C08G 83/00; B01D 71/62; C07H 21/04

(52) U.S. Cl. CPC C08G 83/003; B01D 71/62; C07H 21/04

(57) ABSTRACT

A membrane-assisted process for the preparation of defined monomer sequence polymers, including oligonucleotides and peptides, in solution phase is described. The growing defined monomer sequence polymer is attached to a soluble synthesis support having properties that allow the polymer to be straightforwardly prepared in certain industry-favoured solvents with improved membrane flux and reduced fouling.

### INTRODUCTION

The present invention relates to the preparation of defined monomer sequence polymers, including oligonucleotides and peptides, in solution phase. In particular, the present invention relates to the preparation of defined monomer sequence polymers, particularly oligonucleotides, by membrane filtration (e.g. diafiltration) processes.

### BACKGROUND OF THE INVENTION

The term “defined monomer sequence polymer” is used in the art to describe a polymer comprising at least two monomers in which at least two of the monomers are distinct from each other and in which the monomers are present in the same order in the polymer chain for all molecules of the polymer. Defined monomer sequence polymers include peptides and oligonucleotides, as well as chemically modified peptides and oligonucleotides, all of which are biologically important molecules and comprise polymers made up of distinct repeat units. In the case of peptides the repeat units are amino acids or their derivatives, while in the case of oligonucleotides the repeat units are nucleotides or their derivatives.

Oligonucleotides (oligos) have recently been validated as a new pharmaceutical modality for treating a wide range of serious or life-threatening indications. Oligos are defined monomer sequence polymers formed from a backbone of ribose phosphate monomers, with each monomer having a variable nucleobase side chain; the building block unit of ribose phosphate bound to a nucleobase constitutes a nucleotide (Nt). The precise sequence of nucleotides defines the oligo's biological function.

The industry standard method for preparing oligos is by solid phase oligo synthesis (SPOS). In SPOS the oligos are synthesised tethered to an insoluble solid support in the form of glass or polymer resin beads. Nucleotide building blocks with a reactive 3′-phosphate moiety are flowed over the solid support. Exposed 5′-hydroxy chain termini couple to the building block extending the growing oligo by one monomer unit. Uncontrolled chain extension is prevented by a temporary protecting group, most commonly 4,4′-dimethoxytriphenylmethyl (DMT, DMTr, or Dmtr). After oligo chain extension is complete the Dmtr is removed by washing the support with acid to expose a new oligo 5′-hydroxy chain terminus so that the cycle can be repeated with a new nucleotide building block. In this way any desired oligo sequence is built up.

A key challenge for SPOS is driving reactions to completion with a limited excess of nucleotide building block. Commercially, nucleotide building blocks are almost universally Dmtr-phosphoramidites. These reagents are costly and chemically unstable to acid, water and oxidation. Therefore, there is a strong economic drive to minimise the excess of building block required to drive chemical chain extension of growing oligos to completion, especially at scales over 100 g per batch.

The maximum scale of oligo preparation that SPOS can achieve is approximately 15 Kg of crude oligo per batch, but for a major medical indication, such as cardiovascular disease, tonnes per annum of oligo would be required that make producing batches of 100 kg or more desirable.

Recent strategies to improve the pharmacokinetics and oral availability of peptide drugs have created a renaissance in peptide therapeutics. With more than 50 peptide drugs approved for clinical use, and many in the development pipeline, global peptide therapeutic demand is rising. Methods to produce peptides industrially include Solid Phase Peptide Synthesis (SPPS).

There is considerable interest in further classes of defined monomer sequence polymers for applications in healthcare 1.2 and for applications in further industries including flat panel displays where characteristics such as the optical properties of conjugated polymers are important.

Liquid phase reactions and liquid phase material handling are established technologies that can be performed at the multi-tonne scale. Therefore, liquid phase synthesis is a strong candidate for manufacture of defined monomer sequence polymers at scale.

One approach to liquid phase synthesis of defined monomer sequence polymers is to carry out sequential reactions, adding monomers to a growing polymer in solution in a step-wise fashion, and then to use a suitable separation technology to separate unreacted monomers from the growing defined monomer sequence polymer.

In liquid phase synthesis all species stay in solution. This is challenging for oligos because, although they have good solubility in several polar aprotic solvents, relatively few of these polar aprotic solvents are compatible with phosphoramidite coupling reactions³ and/or the subsequent acidolysis of the Dmtr protecting group (“detritylation”). Consequently, for most liquid phase oligonucleotide synthesis (LPOS) the aim has been to keep the growing oligo dissolved in acetonitrile⁴, and mixtures thereof, which has long been established as the preeminent solvent for SPOS. Dichloromethane is another viable solvent for LPOS⁵ but is not favoured at industrial scales in many countries due to its environmental impact.

One LPOS strategy⁶ has proposed using linear PEG to maintain solubility of the growing oligo in acetonitrile solution. More recently this observation was extended to DNA 20-mers grown on a PEG-star support⁷, although this latter was not an LPOS method as the oligo was not designed to be detached from the support.

In the above LPOS strategies and others^{4, 21} the supported oligo was separated from reaction debris that might interfere in subsequent cycles of chain extension by precipitation. Precipitation is a challenging procedure to make routine and practical, necessitating that filter cakes do not block the filter bed and can be efficiently washed with solvent. Because LPOS is an iterative process, and pharmaceutical products often require that oligos extend to 20-mers and beyond, it is critical that the precipitation process is highly efficient and reproducible. Bonora's HELP process⁶ necessitates one precipitation for each step of the chain extension cycle, including capping (the blocking of unreacted 5′-hydroxyls by acetylation), meaning that 87 diethyl ether precipitations were required to achieve a 20-mer. Walther et al.⁷ were able to compress their process to just one precipitation per cycle on a 4-arm PEG-star, but at the cost of an average recovery of oligo-star of only 94% per cycle, up to 11-mer. For oligos longer than 11-mer each stage required double precipitation, and the use of a DMSO/acetonitrile mixture for solubility.

Yet another approach to sequential synthesis of oligos uses enzymatic synthesis with suitable monomers including nucleoside triphosphates combined with protecting groups⁸. Such enzymatic processes may be performed in aqueous solvent.

The synthesis of peptides using polymers to support the growing peptide chains has also been reported, with precipitation and liquid-liquid extraction used for separation of growing peptides from unreacted monomers and reaction debris⁹.

An alternative to strategies based on precipitation or liquid-liquid extraction is to use a membrane filtration separation after coupling of a monomer onto a defined monomer sequence polymer to separate unreacted monomers and reaction debris from the growing polymer. The use of membrane separation for iterative synthesis of defined monomer sequence polymers including peptides, oligonucleotides, and polyethylene glycols has been described in the prior art¹⁰⁻¹⁹. However, a number of these processes suffer limitations associated with poor solubility of the growing polymer, membrane fouling and/or unacceptably low membrane flux.

The present invention was devised with the foregoing in mind.

### SUMMARY OF THE INVENTION

According to a first aspect of the present invention there is provided a solution-phase process for the preparation of a first compound being a defined monomer sequence polymer, the process comprising the steps of:
a) growing the first compound by performing one or more sequential coupling reactions, and
b) performing membrane filtration to isolate the growing first compound;
   wherein during steps a) and b), a plurality of growing first compounds are each attached at one end to a soluble synthesis support comprising:
a central hub, and
one or more solubility-enhancing polymers, each attached to the central hub;

wherein the total molecular weight of the one or more solubility-enhancing polymers present within each molecule of soluble synthesis support is ≥9000 Da, and the ratio of the total molecular weight of the one or more solubility-enhancing polymers to the total molecular weight of the plurality of first compounds during steps a) and b) is ≥0.7.

It is particularly suitable that the first compound is an oligonucleotide.

According to a second aspect of the present invention, there is provided a first compound obtained, directly obtained or obtainable by the process of the first aspect.

### DETAILED DESCRIPTION OF THE INVENTION

Throughout the entirety of the description and claims of this specification, where subject matter is described herein using the term “comprise” (or “comprises” or “comprising”), the same subject matter instead described using the term “consist of” (or “consists of” or “consisting of”) or “consist essentially of” (or “consists essentially of” or “consisting essentially of”) is also contemplated.

Throughout the description and claims of this specification, the singular encompasses the plural unless the context otherwise requires. In particular, where the indefinite article is used, the specification is to be understood as contemplating plurality as well as singularity, unless the context requires otherwise.

Features described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any of the specific embodiments recited herein. The invention extends to any novel one, or any novel combination, of the features disclosed in this specification (including any accompanying claims, abstract and drawings), or to any novel one, or any novel combination, of the steps of any method or process so disclosed.

As described hereinbefore, in a first aspect, the present invention provides a solution-phase process for the preparation of a first compound being a defined monomer sequence polymer, the process comprising the steps of:
a) growing the first compound by performing one or more sequential coupling reactions, and
b) performing membrane filtration to isolate the growing first compound;
   wherein during steps a) and b), a plurality of growing first compounds are each attached at one end to a soluble synthesis support comprising:
a central hub, and
one or more solubility-enhancing polymers, each attached to the central hub;
   wherein the total molecular weight of the one or more solubility-enhancing polymers present within each molecule of soluble synthesis support is ≥9000 Da, and
   the ratio of the total molecular weight of the one or more solubility-enhancing polymers to the total molecular weight of the plurality of first compounds during steps a) and b) is ≥0.7.

Through rigorous investigations, the inventors have devised a vastly improved process for the solution-phase synthesis of defined monomer sequence polymers such as oligonucleotides, peptides and peptide nucleic acids using membrane filtration (e.g. diafiltration) to purify and/or isolate the polymer during synthesis. The process employs a soluble synthesis support having properties tailored to match the characteristics of the target polymer, thereby allowing the polymer to be straightforwardly prepared in certain industry-favoured solvents with improved membrane flux and reduced fouling.

The term “defined monomer sequence polymer” will be familiar to one of skill in the art as referring to a compound comprising at least 2 (but often many more) monomeric units, in which at least 2 of the monomeric units are distinct from one another, and in which the order in which the monomeric units appear within the polymer is identical for all molecules of the polymer. For example, in a defined monomer sequence polymer formed from monomers A, B, C and D, the sequence of monomeric units may be B-B-B-C-A-A-C-D, with this sequence being identical for all molecules of the polymer. Accordingly, it will be understood that the plurality of growing first compounds in steps a) and b) are identical to one another.

Any defined monomer sequence polymer may be prepared using the present process. Suitably, the first compound is an oligonucleotide or a peptide.

Most suitably, the first compound is an oligonucleotide.

Each first compound, once prepared (i.e. fully grown), may have a molecular weight of ≥1000 Da. Suitably, each first compound has a molecular weight of ≥2000 Da. More suitably, each first compound has a molecular weight of ≥3000 Da. Even more suitably, each first compound has a molecular weight of ≥5000 Da. The first compound is suitably an oligonucleotide.

During the process, the first compound is grown from its constituent parts by performing one or more coupling reactions. Each growing first compound is attached at one end to a soluble synthesis support. The nature of this attachment may be direct or indirect (e.g. via a linker).

The soluble synthesis support comprises a central hub and one or more solubility-enhancing polymers, each attached directly or indirectly (e.g. via a linker) to the central hub. Each one of the plurality of first compounds may be attached directly or indirectly (e.g. via a linker) at one of its ends to the central hub, to a solubility-enhancing polymer, or to any linker that may be linking the central hub to a solubility-enhancing polymer. Suitably, each one of the plurality of first compounds is attached directly or indirectly (e.g. via a linker) at one of its ends to a solubility-enhancing polymer. The growing first compound attached to the soluble synthesis support may be referred to herein as the supported growing first compound.

In particular embodiments, the one or more solubility-enhancing polymers are each attached to the central hub and each one of the growing first compounds is attached at one of its ends to a solubility-enhancing polymer. Suitably, within each molecule of soluble synthesis support, the number of solubility-enhancing polymers is equal to the number of growing first compounds.

Where each first compound is attached to a solubility-enhancing polymer via a linker, a range of chemistries is available to construct this linkage. For example, in the context of oligonucleotide synthesis, if the solubility-enhancing polymer terminates with hydroxyl functionality, this can be esterified with a nucleoside succinate. If the solubility-enhancing polymer terminates with an amino functionality this can be condensed directly with a nucleoside succinate to form a succinate ester-amide. Other linkages may have greater stability; for instance, a PEG-amine can be reacted with Fmoc-sarcosine, or Boc-sarcosine, then deprotected to leave a secondary N-methyl poly(ethylene glycol) chain terminus. Scheme 1 below illustrates various suitable linkers:

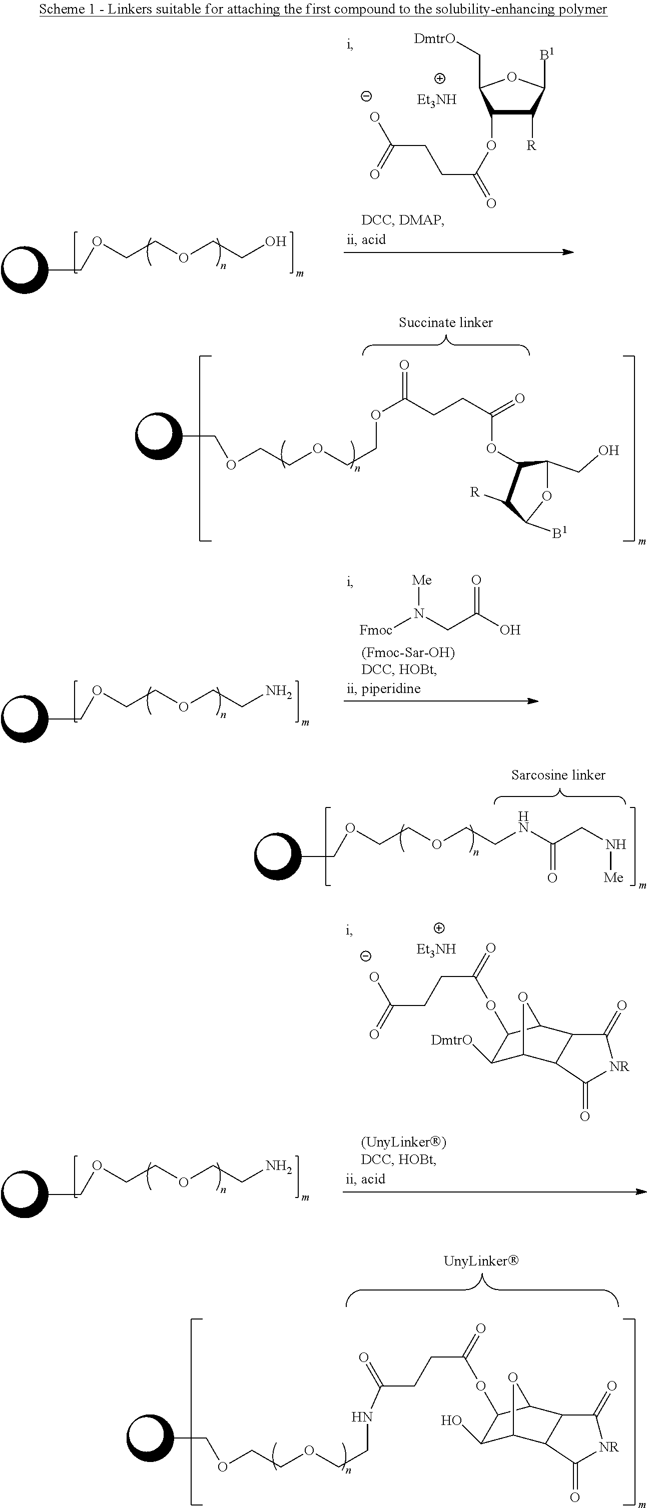

Alternatively, when step a) involves preparing an oligonucleotide via phosphoramidite chemistry, a PEG-amine may be reacted with one of the “universal” linkers. Universal linkers are desirable because the support can be loaded directly with a nucleoside phosphoramidite of choice (but liberating the hydroxy terminal oligo during global deprotection) without recourse to a separate nucleoside succinate building block. After loading onto the solubilising support a temporary hydroxyl protecting group, usually Dmtr, is unblocked ready to participate in the oligo chain extension cycle.

Suitably, each one of the growing first compounds is attached at one of its ends to a solubility-enhancing polymer via a linker having a molecular weight of <600 Da. More suitably, the linker has a molecular weight of <300 Da. A particularly suitable linker is a sarcosine succinate linker. In such embodiments, the first compound is suitably an oligonucleotide and the one or more solubility-enhancing polymers is suitably poly(ethylene glycol).

In particular embodiments, the one or more solubility-enhancing polymers are each attached to the central hub and each one of the growing first compounds is attached at one of its ends to a solubility-enhancing polymer via a linker having a molecular weight of <600 Da (e.g. a molecular weight of <300 Da, such as a sarcosine succinate linker); and within each molecule of soluble synthesis support, the number of solubility-enhancing polymers is equal to the number of growing first compounds. In such embodiments, the first compound is suitably an oligonucleotide and the one or more solubility-enhancing polymers is suitably poly(ethylene glycol).

The central hub may take a variety of forms. The central hub may be an atom (e.g. N or C) or an organic moiety (such as a benzene ring), onto which the one or more solubility-enhancing polymers are attached, directly or indirectly. Suitably, the central hub of each soluble synthesis support has a molecular mass of <1500 Da. Most suitably, the central hub of each soluble synthesis support has a molecular mass of <300 Da (e.g. a carbon atom).

The nature of the solubility-enhancing polymer(s) will depend on the nature of the first compound and the solvent in which it is to be prepared. The one or more solubility-enhancing polymers may be selected from the group consisting of poly(alkylene glycols), polyesters, polyamide, vinyl polymers, diene polymers, poly(alkylene imines), poly(amidoamines) and polysiloxanes, non-limiting examples of which include poly(ethylene glycol) (PEG), poly(propylene glycol) (PPG), poly(butylene glycol), poly(dimethylsiloxane) (PDMS), polybutadiene, polyisoprene, polystyrene, nylon, poly(ethylene imine) (PEI), poly(propylene imine), poly(L-lysine) (PLL), poly(methyl methacrylate) (PMMA), poly(vinyl benzoic acid), poly(hydroxystyrene), N-substituted glycines, and poly(lactide-co-glycolide) (PLGA). Suitably, the one or more solubility-enhancing polymers are selected from the group consisting of poly(alkylene glycols) (e.g. poly(ethylene glycol), polyesters (e.g. poly(lactide-co-glycolide) and polysiloxanes (e.g. polydimethylsiloxane). Even more suitably, the one or more solubility-enhancing polymers are poly(alkylene glycols).

Particularly suitably, the one or more solubility-enhancing polymers are poly(ethylene glycol) (PEG). Poly(ethylene glycol) is highly soluble in acetonitrile, the solvent favoured by industry for coupling nucleotides to prepare oligonucleotides (e.g. phosphoramidite couplings to prepare oligonucleotides).

The one or more solubility-enhancing polymers may be a poly(ethylene glycol) derivative, such as poly(propylene glycol), poly(ether amines) (e.g. Jeffamine® or Elastamine®) (H₂NCHMeCH₂(OCHMeCH₂)ₓ(OCH₂CH₂)_{y}OMe), which are commercially available in a range of lengths (small to large x+y) and hydrophobicities (x/y large=hydrophobic; x/y small=hydrophilic).

The one or more solubility-enhancing polymers may be a plurality (e.g. 2-10) of solubility-enhancing polymers (i.e. each molecule of soluble synthesis support may comprise a plurality (e.g. 2-10) of solubility-enhancing polymers). Suitably, the one or more solubility-enhancing polymers is 2-8 solubility-enhancing polymers. Most suitably, the one or more solubility-enhancing polymers is 3-4 solubility-enhancing polymers (e.g. 3-4 molecules of poly(ethylene glycol)).

In particular embodiments, each molecule of soluble synthesis support comprises 3-4 solubility-enhancing polymers (e.g. poly(ethylene glycol)), each attached to the central hub, and each one of the growing first compounds is attached at one of its ends to a solubility-enhancing polymer. Suitably, within each molecule of soluble synthesis support, the number of solubility-enhancing polymers is equal to the number of growing first compounds.

The total molecular weight of the one or more solubility-enhancing polymers present within each molecule of soluble synthesis support is ≥9000 Da. As used herein, the molecular weight of a given solubility-enhancing polymer refers to the mass of the polymeric (i.e. repeating) portion of the polymer. By way of example, for a poly(ethylene glycol) solubility-enhancing polymer that is attached to the central hub at one end and to the growing first compound at the other end, the molecular weight of the solubility-enhancing polymer is the mass of all-[CH₂CH₂O]-repeating units. For illustrative purposes, each molecule of soluble synthesis support may contain a carbon atom (as central hub) attached directly or indirectly to 4 poly(ethylene glycol) polymers (serving as solubility-enhancing polymers), where each poly(ethylene glycol) polymer has a molecular weight of 2500 Da (approximately 57 repeating-[CH₂CH₂O]-units) (i.e. a 10 kDa 4-arm PEG star support). Alternatively, each molecule of soluble synthesis support may contain a benzene ring (as central hub) attached directly or indirectly to 3 poly(ethylene glycol) polymers (serving as solubility-enhancing polymers), where 2 of the 3 poly(ethylene glycol) polymers each have a molecular weight of 4000 Da (approximately 91 repeating —[CH₂CH₂O]-units), and the third poly(ethylene glycol) polymer has a molecular weight of 2000 Da (approximately 45 repeating —[CH₂CH₂O]— units). The inventors have determined that soluble synthesis supports meeting this minimum molecular weight requirement allow first compounds, particularly those of high molecular weight, to be more straightforwardly prepared in certain industry-favoured solvents with improved membrane flux and reduced fouling.

The total molecular weight of the one or more solubility-enhancing polymers present within each molecule of soluble synthesis support may be ≥9500 Da. Suitably, the total molecular weight of the one or more solubility-enhancing polymers present within each molecule of soluble synthesis support is ≥10,000 Da. More suitably, the total molecular weight of the one or more solubility-enhancing polymers present within each molecule of soluble synthesis support is ≥15,000 Da. Even more suitably, the total molecular weight of the one or more solubility-enhancing polymers present within each molecule of soluble synthesis support is ≥20,000 Da. Yet even more suitably, the total molecular weight of the one or more solubility-enhancing polymers present within each molecule of soluble synthesis support is ≥30,000 Da. Most suitably, the total molecular weight of the one or more solubility-enhancing polymers present within each molecule of soluble synthesis support is ≥35,000 Da.

The one or more solubility-enhancing polymers may be a plurality (e.g. 2-10) of solubility-enhancing polymers, each having a molecular weight of ≥2000 Da, or each having a molecular weight of ≥2250 Da, or each having a molecular weight of ≥4000 Da, or each having a molecular weight of ≥8000 Da. Suitably, the one or more solubility-enhancing polymers are poly(ethylene glycol). Within each molecule of soluble synthesis support, the number of solubility-enhancing polymers may be equal to the number of growing first compounds.

The one or more solubility-enhancing polymers may be 2-8 solubility-enhancing polymers, each having a molecular weight of ≥2000 Da, or each having a molecular weight of ≥2250 Da, or each having a molecular weight of ≥4000 Da, or each having a molecular weight of ≥8000 Da. Suitably, the one or more solubility-enhancing polymers are poly(ethylene glycol). Within each molecule of soluble synthesis support, the number of solubility-enhancing polymers may be equal to the number of growing first compounds.

The one or more solubility-enhancing polymers may be 3-4 solubility-enhancing polymers, each having a molecular weight of ≥2000 Da, or each having a molecular weight of ≥2250 Da, or each having a molecular weight of ≥4000 Da, or each having a molecular weight of ≥8000 Da. Suitably, the one or more solubility-enhancing polymers are poly(ethylene glycol). Within each molecule of soluble synthesis support, the number of solubility-enhancing polymers may be equal to the number of growing first compounds.

In particular embodiments, each molecule of soluble synthesis support comprises 4 poly(ethylene glycol) polymers, each poly(ethylene glycol) polymer having a molecular weight of 2300-2800 Da. Suitably, each molecule of soluble synthesis support comprises 4 growing first compounds, each one being attached at one end to a poly(ethylene glycol) polymer. For example, the soluble synthesis support may be a 10 kDa 4-arm PEG star, a term used herein to denote a support comprising 4 PEG chains, each of 2500 Da, radiating out from a carbon atom acting as central hub. The first compound is suitably an oligonucleotide.

In particular embodiments, each molecule of soluble synthesis support comprises 4 poly(ethylene glycol) polymers, each poly(ethylene glycol) polymer having a molecular weight of 4000-6000 Da. Suitably, each molecule of soluble synthesis support comprises 4 growing first compounds, each one being attached at one end to a poly(ethylene glycol) polymer. For example, the soluble synthesis support may be a 20 kDa 4-arm PEG star, a term used herein to denote a support comprising 4 PEG chains, each of 4000-6000 Da, radiating out from a carbon atom acting as central hub. The first compound is suitably an oligonucleotide.

In other particular embodiments, each molecule of soluble synthesis support comprises 4 poly(ethylene glycol) polymers, each poly(ethylene glycol) polymer having a molecular weight of 8000-12,000 Da. Suitably, each molecule of soluble synthesis support comprises 4 growing first compounds, each one being attached at one end to a poly(ethylene glycol) polymer. For example, the soluble synthesis support may be a 40 kDa 4-arm PEG star, a term used herein to denote a support comprising 4 PEG chains, each of 10 kDa, radiating out from a carbon atom acting as central hub.

The process may be such that the first compound is an oligonucleotide; each molecule of soluble synthesis support comprises a plurality (e.g. 3-4) of solubility-enhancing polymers, each attached to the central hub, said polymers being poly(ethylene glycol); and within each molecule of soluble synthesis support, the number of solubility-enhancing polymers is equal to the number of growing first compounds.

Each molecule of soluble synthesis support suitably has a structure according to Formula I below:

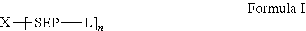

wherein
X represents the central hub (e.g. a carbon atom);
SEP represents a solubility-enhancing polymer (e.g. poly(ethylene glycol));
L is absent or a linker (e.g. an organic moiety having a molecular weight of <600 Da or <300 Da); and
n is 2-8 (e.g. 3-4).

Each one of the plurality of growing first compounds may be attached at one of its ends to L (when present), to SEP or to X. Suitably, each one of the plurality of growing first compounds is attached at one of its ends to L. Where the first compound is an oligonucleotide and SEP is poly(ethylene glycol), L may be a sarcosine succinate linker.

In particular embodiments, X is a carbon atom, SEP is poly(ethylene glycol), L is an organic moiety having a molecular weight of <600 Da (such as a molecular weight of <300 Da, e.g. a sarcosine succinate linker, or another linker mentioned hereinbefore) and n is 3-4. Suitably, each poly(ethylene glycol) has a molecular weight of ≥2000 Da. More suitably, each poly(ethylene glycol) has a molecular weight of ≥2250 Da. Even more suitably, each poly(ethylene glycol) has a molecular weight of ≥4000 Da or ≥8000 Da. Within each molecule of soluble synthesis support, the number of solubility-enhancing polymers is suitably equal to the number of growing first compounds.

In step a), the first compound is grown by performing one or more coupling reactions. The coupling reactions may be monomeric, dimeric or oligomeric in nature. For example, when the first compound being prepared is an oligonucleotide, a coupling reaction may involve adding a single nucleotide (i.e. a monomeric building block) to each growing first compound. Alternatively, when the first compound being prepared is an oligonucleotide, a coupling reaction may involve adding a dimer (i.e. a building block consisting of 2 pre-coupled nucleotides) to each growing first compound. Alternatively still, when the first compound being prepared is an oligonucleotide, a coupling reaction may involve adding an oligomer (i.e. a building block consisting of 3 or more pre-coupled nucleotides) to each growing first compound.

In its simplest sense, step a) may comprise only a single coupling reaction, for example between an initial monomeric unit already bound to the soluble synthesis support and a further monomeric, dimeric or oligomeric building block. Suitably, step a) comprises performing two or more sequential coupling reactions. More suitably, step a) comprises performing four or more sequential coupling reactions. Even more suitably, step a) comprises performing six or more sequential coupling reactions. Yet more suitably, step a) comprises performing ten or more sequential coupling reactions. Most suitably, step a) comprises performing fifteen or more sequential coupling reactions.

Each coupling reaction typically involves reacting a free (unprotected) terminal of a growing first compound with a reactive terminal of a monomer, dimer or oligomer to be coupled, and subsequently deprotecting the terminal of the newly coupled monomer, dimer or oligomer to generate a new free (unprotected) terminal (in preparation for performing a subsequent coupling reaction). As discussed hereinbefore, the skilled person will be familiar with protecting groups used to prevent uncontrolled polymer chain extension in the solution-phase synthesis of defined monomer sequence polymers such as oligonucleotides and peptides, as well as the manner in which they can be removed.

Step a) is suitably conducted in at least one organic solvent. More suitably, step a) is conducted in acetonitrile, optionally mixed with another organic solvent. Most suitably, step a) is conducted in neat acetonitrile. Acetonitrile is the solvent favoured by industry for coupling nucleotides to prepare oligonucleotides.

In particular embodiments, the first compound is an oligonucleotide, and step a) comprises growing the oligonucleotide by performing a plurality of coupling reactions. Each coupling reaction suitably involves the sequential addition of nucleotides (or nucleosides), dinucleotides and/or oligonucleotides to the growing first compound. As described hereinbefore, the skilled person will be familiar with techniques for the stepwise growth of oligonucleotides, e.g. by sequential coupling of phosphoramidite monomers using the Dmtr group for 5′ hydroxy protection. For example, after loading of the soluble synthesis support with a first nucleotide/nucleoside monomer and performing deprotection to expose a free hydroxyl of the loaded monomer, chain extension is performed using conventional phosphoramidite chemistry, followed by sulfur transfer or oxidation of the internucleotide linkage. With a suitable choice of permanent protecting groups (i.e. those that are only removed during global deprotection) on the nucleotide or nucleoside, and a compatible linker chemistry, other classes of chemistry (e.g. phosphotriester, H-phosphonate, Baran chemistry²², etc.) are also compatible with the soluble synthesis supports described herein.

Step b) involves performing membrane filtration (e.g. diafiltration) to isolate the growing first compound prepared in step a). Membrane filtration may be performed to separate the supported growing first compound from a reaction by-product formed as part of a coupling reaction (e.g. a protecting group cleaved from the terminal of the growing first compound) or from an excess reagent used as part of a coupling reaction (e.g. an excess of a monomeric, dimeric or oligomeric building block to be coupled). The supported growing first compound, excess reagent and reaction by-product remain in solution during steps a) and b).

Membrane filtration may therefore be performed once or twice for a given coupling reaction. A first filtration may involve separating the supported growing first compound from a reaction by-product formed as part of a coupling reaction (e.g. a protecting group cleaved from the terminal of the growing first compound). A second filtration may involve separating the supported growing first compound from an excess reagent used as part of a coupling reaction (e.g. an excess of a monomeric, dimeric or oligomeric building block to be coupled). Suitably, membrane filtration is performed twice per coupling reaction.

Membrane filtration need not be performed as part of every sequential coupling reaction conducted as part of step a). For example, if step a) comprises growing a first compound by performing 3 sequential coupling reactions, membrane filtration may be performed as part of only 1 or 2 of these reactions.

In particular embodiments, membrane filtration is performed as part of every sequential coupling reaction conducted as part of part step a). Suitably, for each sequential coupling reaction, membrane filtration is performed twice (e.g. as described hereinbefore).

Steps a) and b) may be performed in the same or different solvents. Suitably, steps a) and b) are performed in the same solvent (e.g. at least one organic solvent, such as acetonitrile). Most suitably, steps a) and b) are performed in acetonitrile.

The process may be such that step b) involves performing membrane filtration (e.g diafiltration) as part of every sequential coupling reaction conducted as part of part step a); for each coupling reaction, membrane filtration is performed to (i) separate the supported growing first compound from a reaction by-product formed as part of a coupling reaction (e.g. a protecting group cleaved from the terminal of the growing first compound), and (ii) separate the supported growing first compound from an excess reagent used as part of a coupling reaction (e.g. an excess of a monomeric, dimeric or oligomeric building block to be coupled); and steps a) and b) are conducted in the same solvent (e.g. an organic solvent).

The inventors have determined that the molecular weight of the solubility-enhancing polymer(s) relative to that of the growing first compound plays a key role in allowing the first compound to be straightforwardly prepared in certain industry-favoured solvents with improved membrane flux and reduced fouling. Accordingly, throughout the process of preparing the first compound, the ratio of the total molecular weight of the one or more solubility-enhancing polymers to the total molecular weight of the plurality of growing first compounds in each molecule of soluble synthesis support (also referred to herein as the polymer-first compound ratio) is ≥0.7. For example, in a soluble synthesis support comprising 4 poly(ethylene glycol) polymers serving as solubility-enhancing polymers, each having a mass of 2500 Da (i.e. a total molecular weight 10 kDa per molecule of soluble synthesis support), the total mass of all growing first compounds bound to that soluble synthesis support throughout steps a) and b) never exceeds 14,300 Da (e.g. 3575 Da per growing first compound, assuming there are 4 growing first compounds coupled to the soluble synthesis support). The inventors have determined that observing this ratio dramatically reduces, or avoids altogether, the need for complex solvent mixtures in order to keep the growing first compound in solution during step a). The ability to perform the entirety of steps a) and b) in a single solvent (e.g. acetonitrile) greatly simplifies the overall process, whilst avoiding reductions in membrane flux that can occur when the addition of a cosolvent gives rise to a more viscous solvent mixture.

In calculating the molecular weight of the plurality of growing first compounds, the mass of any permanent protecting groups is included. Permanent protecting groups will be understood to be those that remain present on the growing first compound until it is eventually cleaved from the soluble synthesis support. For example, where the growing first compound is an oligonucleotide, permanent protecting groups may be used to protect the nucleobases and phosphate groups of the oligonucleotide during its synthesis. Permanent protecting groups are distinct from temporary protecting groups (e.g. Dmtr in the case of an oligonucleotide first compound), which may be used to prevent uncontrolled chain extension during growth of the first compound and are cleaved in preparation for the addition of a new building block to be coupled. In calculating the molecular weight of the plurality of growing first compounds, the mass of any temporary protecting groups is not included. The mass of any group(s) that will remain part of the soluble synthesis support once the first compound is eventually cleaved therefrom (e.g. a linker) is also not included.

Suitably, the ratio of the total molecular weight of the one or more solubility-enhancing polymers to the total molecular weight of the plurality of growing first compounds in each molecule of soluble synthesis support is ≥0.8 during steps a) and b). More suitably, the ratio of the total molecular weight of the one or more solubility-enhancing polymers to the total molecular weight of the plurality of growing first compounds in each molecule of soluble synthesis support is ≥0.9 during steps a) and b). Most suitably, the ratio of the total molecular weight of the one or more solubility-enhancing polymers to the total molecular weight of the plurality of growing first compounds in each molecule of soluble synthesis support is ≥1.0 during steps a) and b).

The ratio of the total molecular weight of the one or more solubility-enhancing polymers to the total molecular weight of the plurality of growing first compounds in each molecule of soluble synthesis support may be ≤2.4. The inventors have determined that when the ratio is very high, the viscosity of the reaction solution may become too high, leading to significant reduction in flux caused by high building block rejection, and eventually membrane fouling. Suitably, the ratio of the total molecular weight of the one or more solubility-enhancing polymers to the total molecular weight of the plurality of growing first compounds in each molecule of soluble synthesis support is ≤2.2. Most suitably, ratio of the total molecular weight of the one or more solubility-enhancing polymers to the total molecular weight of the plurality of growing first compounds in each molecule of soluble synthesis support is ≤2.0.

Membrane filtration is suitably membrane diafiltration. More suitably, membrane filtration is organic solvent nanofiltration (OSN).

Suitable membranes for use in step b) include polymeric membranes, ceramic membranes, and mixed polymeric/inorganic membranes. Membrane rejection Rᵢ is a common term known by those skilled in the art and is defined as:

$${R}_{i} = ( 1 - \frac{{C}_{Pi}}{{C}_{Ri}} ) \times 100 \% \qquad eq . ( 1 )$$

where C_{P,i}=concentration of species i in the permeate, permeate being the liquid which has passed through the membrane, and C_{R,i}=concentration of species i in the retentate, retentate being the liquid which has not passed through the membrane. It will be appreciated that a membrane is suitable for the invention if

R_{(supported growing first compound)}>R_{(at least one reaction by-product or reagent)}.

Typically, in step b), the crude mixture comprising the supported growing first compound is pressurised against a size-selective solvent stable membrane. Here, the soluble synthesis support plays a critical role, beyond being a passive solubility aid. During the process of diafiltration (separation of solutes by permeating the solution through a selective membrane) solutes that exhibit any rejection by the membrane accumulate on the retentate side at the interface between the bulk solution and the membrane. The soluble synthesis support is designed to have the highest possible, preferably 100%, rejection by the membrane. Excess reagents used in the coupling reaction(s) (e.g. nucleotides) typically have the next highest molecular weight, such that they typically have the next highest rejection. It is important to remove all excess reagent prior to beginning the next coupling reaction so as to prevent the free hydroxyl group of a retained nucleotide providing a site for unwanted growth of a truncated oligo contaminant.

To achieve the highest possible purity of the fully grown first compound, e.g. an oligonucleotide, the highest possible coupling efficiency is highly desirable. Given that the rate of the bimolecular reaction of a hydroxy terminus of a growing oligonucleotide with a nucleotide building block is approximately proportional to the concentration of both species, the highest practical concentration of both the growing oligonucleotide and building block should be achieved to allow the process to achieve near quantitative conversion. Since a high building block concentration could be used to drive reactions nearer to 100% completion, a large excess of building block may seem advantageous. However, both for economical first compound synthesis (for example, nucleotide building blocks are very costly) and to minimise the amount of debris that must be removed by diafiltration, a low excess of building block is preferred and therefore highly desirable. Furthermore, a low excess of building block minimises the amount of diafiltration solvent required to achieve a specified purity of first compound; typically, 0.1% of the initial concentration of residual building block (=0.001×0.5 equivalents) is acceptable.

As a compromise between these two extremes, when the first compound is an oligonucleotide, a 2 to 15 mM concentration of growing first compound is desirable for rapid coupling in acetonitrile with an economical excess of 1-2 (e.g. 1.5) equivalents of oligonucleotide (e.g. phosphoramidite) building block. LPOS phosphoramidite chain extension reactions can be initiated with common activators, such as ethylthiotetrazole (ETT) or dicyanoimidazole (DCI), and proceed for 5 to 20 minutes. The reaction may be quenched with a small excess of an alcohol or water, then oxidation (camphorsulfonyl oxaziridine (CSO), or tert-butyl hydroperoxide) or sulfur transfer (phenylacetyl disulfide (PADS), or xanthane hydride (XH)) undertaken, after which the crude mixture can be purified by organic solvent nanofiltration.

In this practical range of concentrations, it is found that low molecular weight supports, (e.g. a 2 kDa 3-arm PEG star), particularly those that are not very soluble, have low membrane rejection. It is also observed that the building block rejection rises in the presence of the growing first compound (e.g. growing oligonucleotide), often to unviable levels. For instance with a supported growing first compound rejection of 95%, the residual building block could have as high as 70% rejection, meaning that too much of the supported growing first compound would be lost to the permeate before the building block was sufficiently removed to proceed with the next reaction.

The solubility and rejection of the supported growing first compound can be increased by using a higher molecular weight support, e.g. 10 kDa 4-arm PEG star. It is then found that, in combination with the high rejection (e.g. >98%) of the supported growing first compound (e.g. supported growing oligonucleotide), although the building block (e.g. nucleotide) rejection (20-40%) is still higher than when measured in isolation (5-20%), separation is now possible without losing considerable quantities of supported first compound to the permeate.

Typically, as multiple sequential coupling reactions are carried out, the membrane is found to increase in rejection of both the supported growing first compound and of the building block. After 2 to 5 couplings, excessively large volumes of solvent may need to be permeated to reduce the building block concentration to the level where another coupling is feasible. At this point the fouled membranes may need to be changed to continue the synthesis. Even when the solubility of the supported growing first compound (e.g. oligonucleotide) in acetonitrile is exceeded and a second stronger solvent is added to compensate (e.g. pyridine, dimethylformamide, dimethylsulfoxide, sulfolane), the membrane may still continue to foul during later diafiltration.

The process of the invention, in particular the nature of the soluble synthesis support and its molecular weight relative to that of the growing first compound, serves to mitigate these issues. For example, a 10 kDa 4-arm PEG star (2500 Da per PEG arm) soluble synthesis support is sufficient to carry a growing oligonucleotide through to completion of an 8-mer synthesis with minimal fouling and without the need to add a second, stronger solvent. A 4-arm PEG 40 kDa star soluble synthesis support is able to carry a growing oligonucleotide through to completion of a 20-mer synthesis with minimal fouling and without the need to add a second strong solvent.

The membranes useful in step b) may be formed from any polymeric or ceramic material which provides a separating layer capable of preferentially separating the supported growing first compound from at least one reaction by-product or reagent used in step a). In other words, the membrane will exhibit a rejection for the supported growing first compound that is greater than the rejection for the reaction by-product or reagent. Suitably, the membrane is formed from or comprises a polymeric material suitable for fabricating microfiltration, ultrafiltration, nanofiltration or reverse osmosis membranes, including polyethylene, polypropylene, polytetrafluoroethylene (PTFE), polyvinylidene difluoride (PVDF), polysulfone, polyethersulfone, polyacrylonitrile, polyamide, polyester, polyimide, polyetherimide, cellulose acetate, polyaniline, polypyrrole, polybenzimidazole, polyetheretherketone (PEEK) and mixtures thereof. The membranes can be made by any technique known in the art, including sintering, stretching, track etching, template leaching, interfacial polymerisation or phase inversion. Membranes may be composite in nature (e.g. a thin film composite membrane) and/or be crosslinked or treated so as to improve their stability in the solvent used in step a). PCT/GB2007/050218 and PCT/GB2015/050179 describe membranes which may be suitable for use in step b), and U.S. Pat. No. 10,913,033 describes a membrane particularly useful in step b). The membrane will be stable in the solvent used in step b).

In particular embodiments, the membrane used in step b) is a crosslinked polybenzimidazole membrane (e.g. an integrally skinned, asymmetric, crosslinked polybenzimidazole membrane).

The process may further comprise the step: c) cleaving the first compound, once fully grown, from the soluble synthesis support. Cleaving the fully grown (i.e. full-length) first compound from the soluble synthesis support yields a plurality of molecules of the fully grown first compound (e.g. oligonucleotide).

In particular embodiments, the first compound is an oligonucleotide. The following paragraphs provide further discussion of oligonucleotide first compounds and their derivatives.

In the context of the invention, an oligonucleotide may have at least one backbone modification, and/or at least one sugar modification and/or at least one base modification compared to an RNA or DNA-based oligonucleotide.

The oligonucleotide may contain at least 1 modified nucleotide residue. The modification may be at the 2′ position of the sugar moiety. Sugar modifications in oligonucleotides described herein may include a modified version of the ribosyl moiety, such as 2′-O-modified RNA such as 2′-O-alkyl or 2′-O (substituted)alkyl e.g. 2′-O-methyl, 2′-O-(2-cyanoethyl), 2′-O-(2-methoxy)ethyl (2′-MOE), 2′-O-(2-thiomethyl)ethyl, 2′-O-butyryl, 2′-O-propargyl, 2′-O-allyl, 2′-O-(3-amino) propyl, 2′-O-(3-(dimethylamino) propyl), 2′-O-(2-amino)ethyl, 2′-O-(2-(dimethylamino)ethyl); 2′-deoxy (DNA); 2′-O (haloalkoxy)methyl (Arai K. et al. Bioorg. Med. Chem. 2011, 21, 6285) e.g. 2′-O-(2-chloroethoxy)methyl (MCEM), 2′-O-(2,2-dichloroethoxy)methyl (DCEM); 2′-O-alkoxycarbonyl e.g. 2′-O-[2-(methoxycarbonyl)ethyl] (MOCE), 2′-O-[2-(N-methylcarbamoyl)ethyl] (MCE), 2′-O-[2-(N, N-dimethylcarbamoyl)ethyl] (DCME); 2′-halo e.g. 2′-F, FANA (2′-F arabinosyl nucleic acid); carbasugar and azasuar modifications; 3′-O-alkyl e.g. 3′-O-methyl, 3′-O-butyryl, 3′-O-propargyl; and their derivatives.

Sugar modifications may be selected from the group consisting of 2′-fluoro (2′-F), 2′-O-methyl (2′-OMe), 2′-O-methoxyethyl (2′-MOE), and 2′-amino. Alternatively, the modification may be 2′-O-MOE. Other sugar modifications include “bridged” or “bicylic” nucleic acid (BNA), e.g. locked nucleic acid (LNA), xylo-LNA, α-L-LNA, β-D-LNA, cEt (2′-O,4′-C constrained ethyl) LNA, cMOEt (2′-0,4′-C constrained methoxyethyl) LNA, ethylene-bridged nucleic acid (ENA), tricyclo DNA; unlocked nucleic acid (UNA); cyclohexenyl nucleic acid (CeNA), altritol nucleic acid (ANA), hexitol nucleic acid (HNA), fluorinated HNA (F-HNA), pyranosyl-RNA (p-RNA), 3′-deoxypyranosyl-DNA (p-DNA); morpholino (as e.g. in PMO, PPMO, PMOPlus, PMO-X); and their derivatives.

Oligonucleotides used in the process of the invention may include other modifications, such as peptide-base nucleic acid (PNA), boron modified PNA, pyrrolidine-based oxy-peptide nucleic acid (POPNA), glycol- or glycerol-based nucleic acid (GNA), threose-based nucleic acid (TNA), acyclic threoninol-based nucleic acid (aTNA), oligonucleotides with integrated bases and backbones (ONIBs), pyrrolidine-amide oligonucleotides (POMs); and their derivatives.

The modified oligonucleotide may comprise a phosphorodiamidate morpholino oligomer (PMO), a locked nucleic acid (LNA), a peptide nucleic acid (PNA), a bridged nucleic acid (BNA) such as (5)-cEt-BNA, or a SPIEGELMER.

Modifications may also be present in the nucleobase. Base modifications include modified versions of the natural purine and pyrimidine bases (e.g. adenine, uracil, guanine, cytosine, and thymine), such as inosine, hypoxanthine, orotic acid, agmatidine, lysidine, 2-thiopyrimidine (e.g. 2-thiouracil, 2-thiothymine), G-clamp and its derivatives, 5-substituted pyrimidine (e.g. 5-methylcytosine, 5-methyluracil, 5-halouracil, 5-propynyluracil, 5-propynylcytosine, 5-aminomethyl uracil, 5-hydroxymethyl uracil, 5-aminomethylcytosine, 5-hydroxymethylcytosine, Super 5 T), 2,6-diaminopurine, 7-deazaguanine, 7-deazaadenine, 7-aza-2, 6-diaminopurine, 8-aza-7-deazaguanine, 8-aza-7-deazaadenine, 8-aza-7-deaza-2, 6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), or derivatives thereof; and degenerate or universal bases, like 2,6-difluorotoluene or absent bases like abasic sites (e.g. 1-deoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683,173. cPent-G, cPent-AP and Pr-AP were shown to reduce immunostimulatory effects when incorporated in SiRNA²⁰.

The nucleobase modification may be selected from the group consisting of 5-methyl pyrimidines, 7-deazaguanosines and abasic nucleotides. Alternatively, the modification may be a 5-methyl cytosine.

Oligonucleotides used in the process of this invention may include a backbone modification, e.g. a modified version of the phosphodiester present in RNA, such as phosphorothioate (PS), phosphorodithioate (PS2), phosphonoacetate (PACE), phosphonoacetamide (PACA), thiophosphonoacetate, thiophosphonoacetamide, phosphorothioate prodrug, H-phosphonate, methylphosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methylboranophosphonothioate, and their derivatives. Another modification includes phosphoramidite, phosphoramidate, N3′˜PS' phosphoramidate, phosphordiamidate, phosphorothiodiamidate, sulfamate, dimethylenesulfoxide, sulfonate, triazole, oxalyl, carbamate, methyleneimino (MMI), and thioacetamido nucleic acid (TANA); and their derivatives.

Backbone modifications may be selected from the group consisting of: phosphorothioate (PS), phosphoramidate (PA) and phosphorodiamidate. The modified oligonucleotide may be a phosphorodiamidate morpholino oligomer (PMO). A PMO has a backbone of methylenemorpholine rings with phosphorodiamidate linkages.

The oligonucleotide may have a phosphorothioate (PS) backbone.

The oligonucleotide may comprise a combination of two or more modifications as described above. A person skilled in the art will appreciate that there are many synthetic derivatives of oligonucleotides.

The first compound may be a gapmer. The 5′ and 3′ wings of the gapmer may comprise or consist of 2′-MOE modified nucleotides. The gap segment of the gapmer may comprise or consist of nucleotides containing hydrogen at the 2′ position of the sugar moiety, i.e. is DNA-like. For example, the 5′ and 3′ wings of the gapmer may consist of 2′-MOE modified nucleotides and the gap segment of the gapmer may consist of nucleotides containing hydrogen at the 2′ position of the sugar moiety (i.e. deoxynucleotides). Alternatively, the 5′ and 3′ wings of the gapmer may consist of 2′-MOE modified nucleotides and the gap segment of the gapmer may consist of nucleotides containing hydrogen at the 2′ position of the sugar moiety (i.e. deoxynucleotides) and the linkages between all of the nucleotides are phosphorothioate linkages.

The following numbered statements 1 to 49 are not claims, but instead describe particular aspects and embodiments of the invention:
1. A solution-phase process for the preparation of a first compound being a defined monomer sequence polymer, the process comprising the steps of:
   a) growing the first compound by performing one or more sequential coupling reactions,
   and
   b) performing membrane filtration to isolate the growing first compound;
wherein during steps a) and b), a plurality growing first compounds are each attached at one end to a soluble synthesis support comprising:
   a central hub, and
   one or more solubility-enhancing polymers, each attached to the central hub;
wherein the total molecular weight of the one or more solubility-enhancing polymers present within each molecule of soluble synthesis support is ≥9000 Da, and the ratio of the total molecular weight of the one or more solubility-enhancing polymers to the total molecular weight of the plurality first compounds during steps a) and b) is ≥0.7.
2. The solution-phase process of statement 1, wherein the total molecular weight of the one or more solubility-enhancing polymers present within each molecule of soluble synthesis support is ≥9500 Da.
3. The solution-phase process of statement 1 or 2, wherein the total molecular weight of the one or more solubility-enhancing polymers present within each molecule of soluble synthesis support is ≥10,000 Da.
4 The solution-phase process of statement 1, 2 or 3, wherein the total molecular weight of the one or more solubility-enhancing polymers present within each molecule of soluble synthesis support is ≥20,000 Da.
5. The solution-phase process of any one of the preceding statements, wherein the total molecular weight of the one or more solubility-enhancing polymers present within each molecule of soluble synthesis support is ≥30,000 Da.
6. The solution-phase process of any one of the preceding statements, wherein the total molecular weight of the one or more solubility-enhancing polymers present within each molecule of soluble synthesis support is ≥35,000 Da.
7. The solution-phase process of any one of the preceding statements, wherein the ratio of the total molecular weight of the one or more solubility-enhancing polymers to the total molecular weight of the plurality of first compounds is ≥0.8.
8. The solution-phase process of any one of the preceding statements, wherein the ratio of the total molecular weight of the one or more solubility-enhancing polymers to the total molecular weight of the plurality of first compounds is ≥0.9.
9. The solution-phase process of any one of the preceding statements, wherein the ratio of the total molecular weight of the one or more solubility-enhancing polymers to the total molecular weight of the plurality of first compounds is ≥1.0.
10. The solution-phase process of any one of the preceding statements, wherein the one or more solubility-enhancing polymers are selected from the group consisting of poly(alkylene glycols) (e.g. poly(ethylene glycol), polyester (e.g. poly(lactide-co-glycolide) and polysiloxanes (e.g. polydimethylsiloxane).
11. The solution-phase process of any one of the preceding statements, wherein the one or more solubility-enhancing polymers are poly(ethylene glycol).
12. The solution-phase process of any one of the preceding statements, wherein the one or more solubility-enhancing polymers is a plurality of solubility-enhancing polymers.
13 The solution-phase process of any one of the preceding statements, wherein the one or more solubility-enhancing polymers is 2-8 solubility-enhancing polymers.
14. The solution-phase process of any one of the preceding statements, wherein the one or more solubility-enhancing polymers is 3-4 solubility-enhancing polymers.
15. The solution-phase process of any one of statements 12, 13 and 14, wherein the solubility-enhancing polymers each have a molecular weight of ≥2000 Da (e.g. ≥2250 Da or ≥4000 Da or ≥8000 Da).
16. The solution-phase process of any one of the preceding statements, wherein the plurality of first compounds is 2-8 first compounds.
17. The solution-phase process of any one of the preceding statements, wherein the plurality of first compounds is 3-4 first compounds
18. The solution-phase process of any one of the preceding statements, wherein the number of solubility-enhancing polymers is equal to the number of first compounds.
19. The solution-phase process of any one of the preceding statements, wherein the one or more sequential coupling reactions is one or more sequential monomeric coupling reactions.
20. The solution-phase process of any one of the preceding statements, wherein step a) comprises performing two or more sequential coupling reactions.
21. The solution-phase process of any one of the preceding statements, wherein step a) comprises performing three or more sequential coupling reactions.
22. The solution-phase process of any one of the preceding statements, wherein step a) comprises performing four or more sequential coupling reactions.
23. The solution-phase process of any one of statements 20, 21 and 22, wherein step b) comprises performing membrane filtration after at least two of the sequential coupling reactions.
24. The solution-phase process of any one of statements 20, 21 and 22, wherein step b) comprises performing membrane filtration after each of the sequential coupling reactions.
25. The solution-phase process of any one of the preceding statements, wherein each one of the plurality of molecules of the growing first compound is attached at one end to the central hub.
26. The solution-phase process of any one of statements 1 to 24, wherein each one of the plurality of growing first compounds is attached at one end to the central hub or to a solubility-enhancing polymer, such that one or more of the growing first compounds is attached to the central hub, and one or more of the growing first compounds is attached to a solubility-enhancing polymer.
27. The solution-phase process of any one of statements 12 to 24, wherein each one of the plurality of growing first compounds is attached at one end to a solubility-enhancing polymer.
28. The solution-phase process of any one of the preceding statements, wherein the first compound is an oligonucleotide or a peptide.
29. The solution-phase process of any one of the preceding statements, wherein the first compound is an oligonucleotide.
30. The solution-phase process of any one of the preceding statements, wherein each first compound has a molecular weight of ≥2000 Da.
31. The solution-phase process of any one of the preceding statements, wherein each first compound has a molecular weight of ≥3000 Da.
32. The solution-phase process of any one of the preceding statements, wherein each first compound has a molecular weight of ≥5000 Da.
33. The solution-phase process of any one of the preceding statements, wherein membrane filtration is membrane diafiltration (e.g. organic solvent nanofiltration).
34. The solution-phase process of any one of the preceding statements, wherein membrane diafiltration is performed using a crosslinked poly(benzimidazole) membrane.
35. The solution-phase process of any one of the preceding statements, wherein steps a) and b) are conducted in the same solvent (e.g. acetonitrile).
36. The solution-phase process of any one of the preceding statements, wherein the one or more solubility-enhancing polymers are each attached to the central hub and each one of the growing first compounds is attached at one of its ends to a solubility-enhancing polymer via a linker having a molecular weight of <600 Da (e.g. a molecular weight of <300 Da, such as a sarcosine succinate linker); and within each molecule of soluble synthesis support, the number of solubility-enhancing polymers is equal to the number of growing first compounds.
37. The solution-phase process of any one of the preceding statements, wherein each molecule of soluble synthesis support comprises 4 poly(ethylene glycol) solubility-enhancing polymers, each poly(ethylene glycol) polymer having a molecular weight of 2300-2800 Da, and each molecule of soluble synthesis support comprises 4 growing first compounds, each one being attached at one end to a poly(ethylene glycol) polymer.
38. The solution-phase process of any one of the preceding statements, wherein each molecule of soluble synthesis support comprises 4 poly(ethylene glycol) solubility-enhancing polymers, each poly(ethylene glycol) polymer having a molecular weight of 4000-6000 Da, and each molecule of soluble synthesis support comprises 4 growing first compounds, each one being attached at one end to a poly(ethylene glycol) polymer.
39. The solution-phase process of any one of the preceding statements, wherein each molecule of soluble synthesis support comprises 4 poly(ethylene glycol) solubility-enhancing polymers, each poly(ethylene glycol) polymer having a molecular weight of 8000-12,000 Da, and each molecule of soluble synthesis support comprises 4 growing first compounds, each one being attached at one end to a poly(ethylene glycol) polymer.
40. The solution-phase process of any one of the preceding statements, wherein the central hub of each soluble synthesis support has a molecular mass of <300 Da and the one or more solubility-enhancing polymers are each attached directly to the central hub, the one or more solubility-enhancing polymers being poly(ethylene glycol).
41. The solution-phase process of any one of the preceding statements, wherein each molecule of soluble synthesis support comprises 4 poly(ethylene glycol) solubility-enhancing polymers, and each molecule of soluble synthesis support comprises 4 growing first compounds, each one being attached at one end to a poly(ethylene glycol) polymer via a linker having a molecular weight of <600 Da (e.g. a molecular weight of <300 Da, such as a sarcosine succinate linker); and the central hub of each soluble synthesis support has a molecular mass of <300 Da.
42. The solution-phase process of any one of the preceding statements, further comprising the step:
c) cleaving the first compound, once fully grown, from the soluble synthesis support.
43. The solution-phase process of any one of the preceding statements, wherein the ratio of the total molecular weight of the one or more solubility-enhancing polymers to the total molecular weight of the plurality of first compounds is ≤2.4.
44. The solution-phase process of any one of the preceding statements, wherein the ratio of the total molecular weight of the one or more solubility-enhancing polymers to the total molecular weight of the plurality of first compounds is ≤2.2.
45. The solution-phase process of any one of the preceding statements, wherein the ratio of the total molecular weight of the one or more solubility-enhancing polymers to the total molecular weight of the plurality of first compounds is ≤2.0.
46. The solution-phase process of any one of the preceding statements, wherein the ratio of the total molecular weight of the one or more solubility-enhancing polymers to the total molecular weight of the plurality of first compounds is ≥0.7 and ≤2.4 (or ≤2.2 or ≤2.0).
47. The solution-phase process of any one of the preceding statements, wherein the ratio of the total molecular weight of the one or more solubility-enhancing polymers to the total molecular weight of the plurality of first compounds is ≥0.8 and ≤2.4 (or ≤2.2 or ≤2.0).
48. The solution-phase process of any one of the preceding statements, wherein the ratio of the total molecular weight of the one or more solubility-enhancing polymers to the total molecular weight of the plurality of first compounds is ≥0.9 and ≤2.4 (or ≤2.2 or ≤2.0).
49. The solution-phase process of any one of the preceding statements, wherein the ratio of the total molecular weight of the one or more solubility-enhancing polymers to the total molecular weight of the plurality of first compounds is ≥1.0 and ≤2.4 (or ≤2.2 or ≤2.0).

### EXAMPLES

One or more examples of the invention will now be described, for the purpose of illustration only, with reference to the accompanying figures:

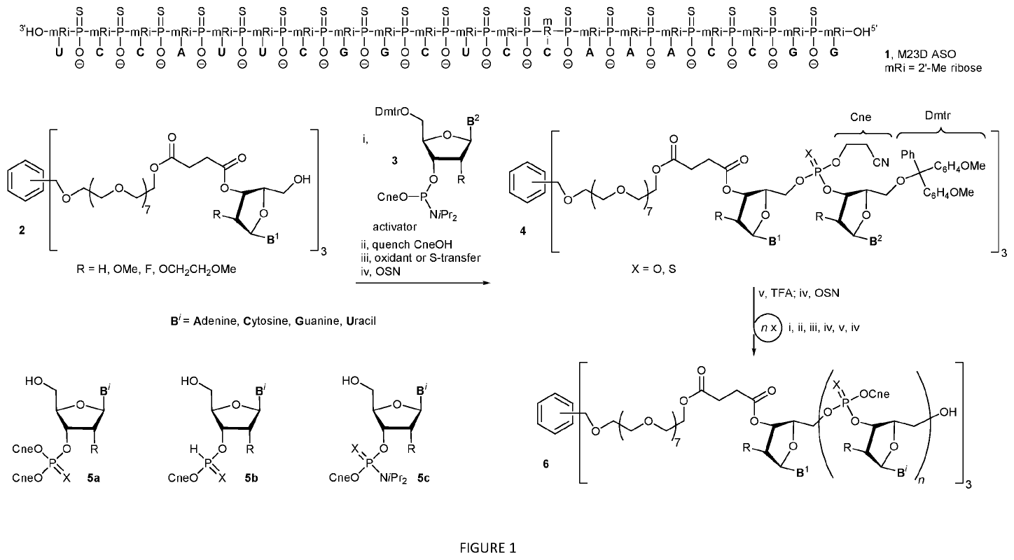

FIG. 1. shows an anti-sense oligo (ASO) M23D, 1, and the synthesis of oligos on a low molecular weight mono-disperse 3-arm star that was used to prepare 1.

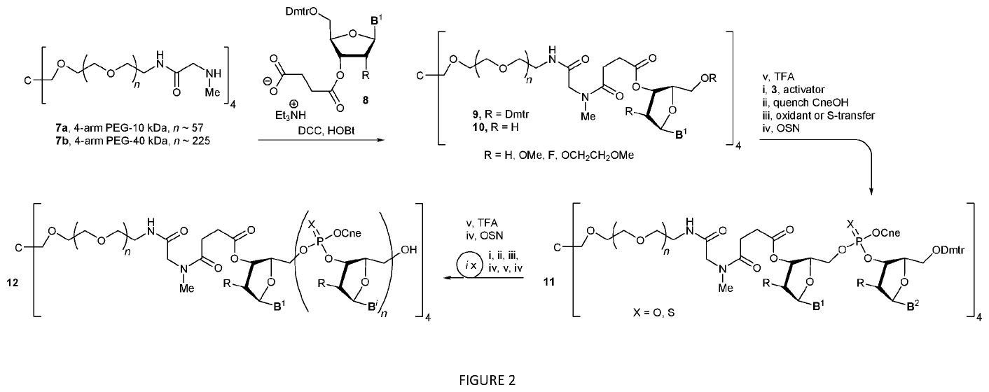

FIG. 2. shows the synthesis of oligos on a) a 10 kDa poly-disperse 4-arm star, and b) a 40 kDa poly-disperse 4-arm star. These supports were both used to prepare model ASO 1.

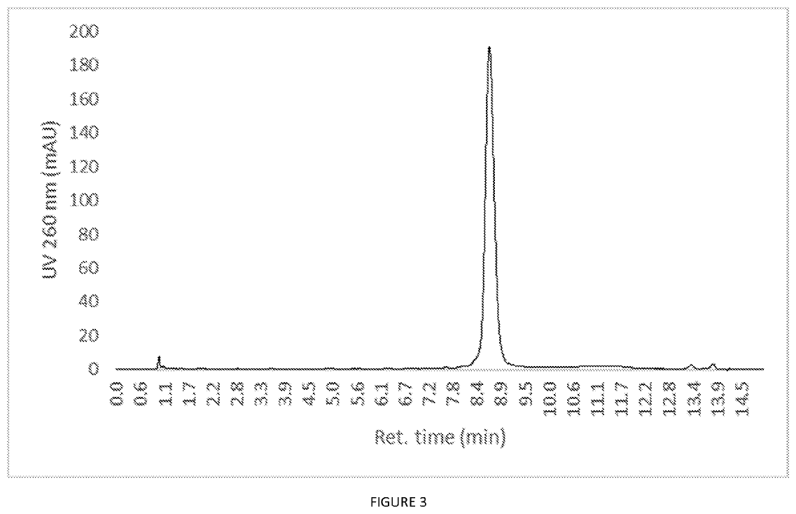

FIG. 3 shows the ion-pair reversed phase (IP-RP) chromatogram, detecting at 260 nm, of fully deprotected 20-mer sequence 1.

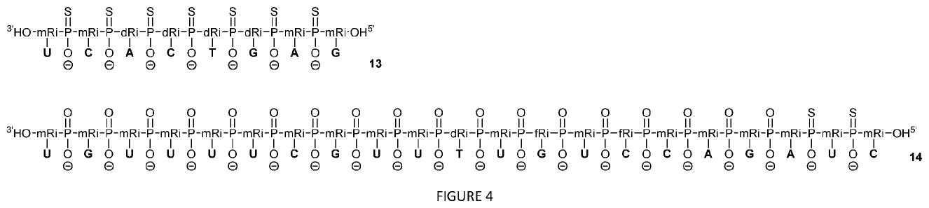

FIG. 4 shows the sequences of test oligos of 8-mer 13, containing deoxy ribonucleotides, and 21-mer 14, with oxygenated internucleotide linkages.

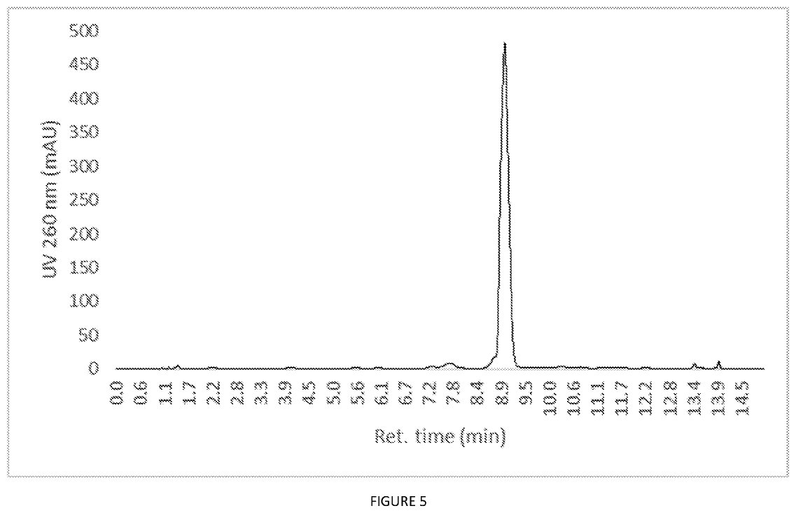

FIG. 5 shows the IP-RP chromatogram (260 nm) of fully deprotected 8-mer sequence 13.

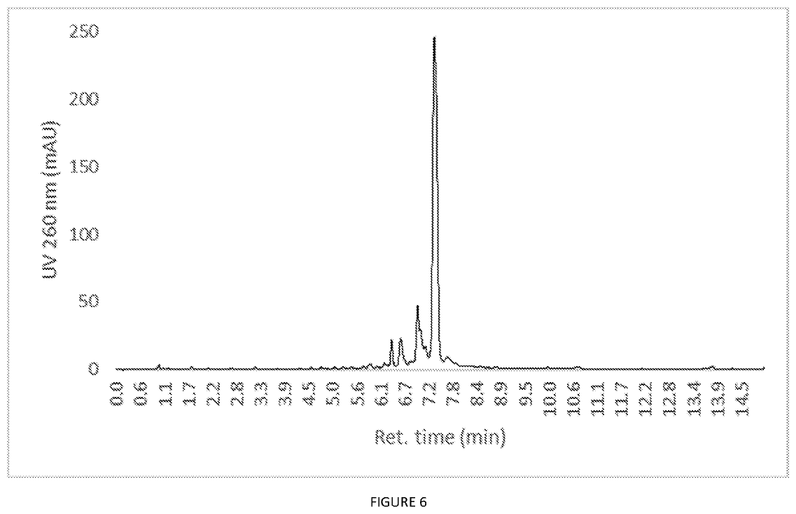

FIG. 6 shows the IP-RP chromatogram (260 nm) of fully deprotected 21-mer sequence 14.

### Comparative Example 1

FIG. 1 shows model anti-sense oligo (ASO) M23D, 1, and the synthesis of oligos on a low molecular weight mono-disperse 3-arm star that was used to prepare 1. The central tris(octagol)-star was loaded with 2′-Me-uridine succinate (8) as described by Gaffney et al.^{14,15} and the loaded support, 2, was reacted with a small excess of phosphoramidite 3 (R=MeO, B²=C^{Ac}, 1.5 eq. per arm) in the presence of ETT. After 20 min the coupling was quenched with CneOH, and the internucleotide linkage was thioylated with PADS. The mixture was transferred to a Nanostar synthesiser fitted with 2 membrane separation stages using PBI18-DBX-M1000 membranes in both stages, and low molecular weight (MW) debris was removed by permeating acetonitrile-sulfolane (1:1 v/v). The resultant partially purified Dmtr-dimer-star was detritylated in the Nanostar synthesiser with 2.5 vol % TFA and cation trap, after which the reaction was quenched with 3-methylpyridine. The mixture was diafiltered once more, but now to complete purity of HO-dimer-star 6 (n=1) with further acetonitrile-sulfolane (1:1 v/v). After detritylation the building block debris, 5, permeates to waste from stage 2 with low rejection of 35-40%.

The coupling cycle was repeated, but now entirely within the Nanostar synthesiser, building up the sequence of ASO 1. As the synthesis cycles advanced the rejection of the oligo-star rose from 99.2 to 99.7%, and the rejection of building block debris after detritylation, 5 (a)-(c), remained in the range 35-40% initially, allowing for effective separation by OSN. However, at 6-mer-star (polymer-first compound ratio of 0.15) the rejection of 5 rose to 45%, and at 7-mer-star to 75% making purification impractical so the membranes were replaced. At Dmtr-8-mer-star the rejection of the oligo-star was 99.8% so synthesiser operation was converted to single membrane stage separation, and the oligo-star rejection was 100% by 10-mer. The rejection of debris 5 maintained a value of 55-60% up to 13-mer-star which allowed for practical separation in a single stage membrane apparatus, but required longer diafiltration. However, at 14-mer-star (polymer-first compound ratio of 0.061) the supported oligo precipitated, necessitating the addition of a third co-solvent to acetonitrile-sulfolane of diglyme to the diafiltration of Dmtr-oligo-star, and pyridine to the HO-oligo-star, to maintain the supported oligo in solution for LPOS. This continued up to full length 20-mer (polymer-first compound ratio of 0.041), but with impractically high rejection of 5 of 75-90%. The oligo-star was then washed from the rig, and diluted into acetonitrile to precipitate the oligo-star which was deprotected by ammonolysis.

### Example 1

FIG. 2a shows the synthesis of ASO 1 on a 10 kDa poly-disperse 4-arm PEG-star. Soluble support 7a was prepared in virtually quantitative yield from commercial 4-arm PEG-10 kDa amine by initial condensation with Fmoc-sarcosine, then treatment of the intermediate with 20% piperidine in DMF. Support 7a was then purified by OSN in acetonitrile. Without further treatment PEG-10k (SarH)₄ 7a was condensed with Dmtr-mU succinate 8 using dicyclohexyl carbodiimide (DCC) and hydroxybenzotriazole (HOBt), then transferred to a single membrane separation stage synthesiser fitted with PBI18-DBX-M1000 membranes, and 9a was diafiltered in neat acetonitrile to remove low MW debris. Detritylation was then performed within the synthesiser, similarly to Comparative Example 1, and diafiltration continued in neat acetonitrile until no remaining succinate building block could be detected.

Loaded nucleoside-star 10a was subjected to chain extension with nucleoside phosphoramidite 3 (R=MeO, B²=C^{Ac}, 1.5 eq. per arm) building block, quenching with CneOH, and finally sulfur transfer, similarly to Comparative Example 1, but now in neat acetonitrile due to the solubilising PEG-star support. Again, after removing low MW reagent debris by OSN (but now in neat acetonitrile), the temporary 5′-Dmtr protecting group was unblocked with 2.5% TFA, the detritylation quenched with excess 3-picoline, and diafiltration continued (in neat acetonitrile) until no building block debris 5 remained. This cycle was repeated, injecting reagents into the synthesiser in the desired sequence to build up ASO 1, and removing excess reagents and debris by OSN. At the 4-mer-star stage (polymer-first compound ratio of 1.53) when the rejection of building block debris rose to >60% after detritylation, a new set of membrane disks was inserted bringing this value down to 35-45% which allows efficient building block removal. Thereafter, the chain extension cycle continued successfully in neat acetonitrile up to 8-mer-star (polymer-first compound ratio of 0.75).

### Comparative Example 2

At the 9-mer-star stage of Example 1 (polymer-first compound ratio of 0.65), an oil separated from the solution after detritylation. At this stage, chain extension could only be continued using a stronger solvent than neat acetonitrile. Acetonitrile was mixed with sulfolane 3:1 to bring the oligo-star back into solution. However, because sulfolane increases the solvent viscosity, this reduced the solvent flux through the membrane by half. After detritylation, the solvent was switched from acetonitrile-sulfolane to acetonitrile-pyridine 4:1 because this mixture is much less viscous than acetonitrile-sulfolane, and diafiltration is faster. However, after every phosphoramidite coupling the solvent was then switched back to acetonitrile-sulfolane because pyridine prevents acid mediated detritylation. The synthesis was continued to full-length 20-mer-star, although at 12-mer (polymer-first compound ratio of 0.47) the sulfolane content during the first diafiltration was increased to acetonitrile-sulfolane 1:1, further reducing flux by half again. The last three cycles of chain extension were very long and required high solvent use to remove the building block debris due to the high (>70%) rejection of the building block debris, but the full length oligo-star was reached and deprotected to afford ASO 1.

### Example 2

FIG. 2b shows the synthesis of ASO 1 on a 40 kDa poly-disperse 4-arm star. Soluble support 7b was prepared in virtually quantitative yield from commercial 4-arm PEG-40 kDa amine by initial condensation with Fmoc-sarcosine, then treatment of the intermediate with 20% piperidine in DMF. Support 7b was then purified by OSN in acetonitrile. Without further treatment PEG-40k (SarH)₄ 7b was condensed with Dmtr-mU succinate 8 using dicyclohexyl carbodiimide (DCC) and hydroxybenzotriazole (HOBt), then transferred to a single membrane separation stage synthesiser fitted with PBI18-DBX-MEA membranes, and 9b was diafiltered in neat acetonitrile to remove low MW debris. Detritylation was then performed within the synthesiser, similarly to Comparative Example 1, and diafiltration continued in neat acetonitrile until no remaining succinate building block could be detected.

Loaded nucleoside-star 10b was subjected to chain extension with nucleoside phosphoramidite 3 (R=MeO, B²=C^{Ac}, 1.5 eq. per arm), quenching with CneOH, and finally sulfur transfer, similarly to Comparative Example 1, but now in neat acetonitrile due to the solubilising PEG-star support. Again, after removing low MW reagent debris by OSN (but now in neat acetonitrile), the temporary 5′-Dmtr protecting group was unblocked with 2.5% TFA, the detritylation quenched with excess 3-picoline, and diafiltration continued (in neat acetonitrile) until no building block debris 5 remained. This cycle was repeated, injecting reagents into the synthesiser in the desired sequence to build up ASO 1. As the length of the oligo on the PEG-star support increased the rejection of the building block debris 5 only increased slowly and the flux also dropped slowly. Eventually the membrane was changed at 11-mer-star (polymer-first compound ratio of 2.15) due to some fouling and the chain extension cycles continued in neat acetonitrile up to full-length 20-mer-star (polymer-first compound ratio of 1.12) without further significant fouling or problematic rise in the building block rejection. The 20-mer-star was washed from the synthesiser and deprotected by ammonolysis to give ASO 1 with 94% UV-purity, FIG. 3.

### Example 3a

FIG. 4 shows 8-mer sequence 13, which was selected as test sequence for the soluble supports because it included deoxyribonucleotide residues. As in Example 1, PEG-10k (SarH)₄ 7a was condensed with Dmtr-mU succinate 8 using dicyclohexyl carbodiimide (DCC) and hydroxybenzotriazole (HOBt), then transferred to a single membrane separation stage synthesiser fitted with PBI17-DBX-M2005 membranes, and 9a was diafiltered in neat acetonitrile to remove low MW debris. Detritylation was then performed within the synthesiser, similarly to Comparative Example 1, and diafiltration continued in neat acetonitrile until no remaining succinate building block could be detected.

Loaded nucleoside-star 10a was subjected to chain extension with nucleoside phosphoramidite 3 (R=MeO, B²=C^{Ac}, 1.5 eq. per arm) building block activated by DCI, quenching with CneOH, and finally sulfur transfer with xanthane hydride dissolved in pyridine, similarly to Example 2 in neat acetonitrile. Again, after removing low MW reagent debris by OSN, the temporary 5′-Dmtr protecting group was unblocked with 5% TFA, the detritylation quenched with excess 3-picoline, and diafiltration continued until no building block debris 5 remained. This cycle was repeated, injecting reagents into the synthesiser in the desired order to build up sequence 13, and removing excess reagents and debris by OSN, except that from this point onwards all detritylation reactions were cooled to between 3 and 5 degrees C. At the 7-mer-star stage (polymer-first compound ratio of 1.85) 20% v/v sulfolane was added and both the synthesis and diafiltration were continued to octamer-star in sulfolane-MeCN 1:4. The 8-mer-star was washed from the synthesiser and deprotected by ammonolysis to give octamer 13 with 94% UV-purity.

### Example 3b

As in Example 2, PEG-40k (SarH)₄ 7b was condensed with Dmtr-mU succinate 8 using dicyclohexyl carbodiimide (DCC) and hydroxybenzotriazole (HOBt), then transferred to a single membrane separation stage synthesiser fitted with PBI16-DBX-M2005 membranes, and 9b was diafiltered in neat acetonitrile to remove low MW debris. Detritylation was then performed within the synthesiser, similarly to Example 3a, and diafiltration continued in neat acetonitrile until no remaining succinate building block could be detected. Loaded nucleoside-star 10b was subjected to chain extension with nucleoside phosphoramidite 3 (R=MeO, B²=C^{Ac}, 1.5 eq. per arm) building block, quenching with CneOH, and finally sulfur transfer, similarly to Example 3a in neat acetonitrile. Again, after removing low MW reagent debris by OSN, the temporary 5′-Dmtr protecting group was unblocked with 5% TFA, the detritylation quenched with excess 3-picoline, and diafiltration continued until no building block debris 5 remained. This cycle was repeated, injecting reagents into the synthesiser in the desired order to build up sequence 13, and removing excess reagents and debris by OSN, except that from this point onwards all detritylation reactions were cooled to between 3 and 5 degrees C., as for Example 3a. As the length of the oligo on the PEG-star support increased the rejection of the building block debris 5 increased slowly and the flux also dropped slowly. Eventually the membrane was changed at 7-mer-star (polymer-first compound ratio of 4.40) due to fouling and the chain extension cycles continued in neat acetonitrile up to full-length 8-mer-star (polymer-first compound ratio of 3.93) although some fouling was experienced due to a rise in building block rejection. The 8-mer-star was washed from the synthesiser and deprotected by ammonolysis to give octamer 13 with 88% UV-purity, FIG. 5.

### Example 4

FIG. 4 shows 21-mer sequence 14, which was selected as a test sequence for the soluble supports because it included PO internucleotide linkages. As in Example 3, PEG-40k (SarH)₄ 7b was condensed with Dmtr-mU succinate 8 using dicyclohexyl carbodiimide (DCC) and hydroxybenzotriazole (HOBt), then transferred to a single membrane separation stage synthesiser fitted with 5 circular cells (52 cm² each) of PBI16-DBX-M2005 membranes, and 9b was diafiltered in neat acetonitrile to remove low MW debris. Detritylation was then performed within the synthesiser, similarly to Example 3a, and diafiltration continued in neat acetonitrile until no remaining succinate building block could be detected.

Loaded nucleoside-star 10b was subjected to chain extension with nucleoside phosphoramidite 3 (R=MeO, B²=C^{Ac}, 1.5 eq. per arm) building block activated with DCI, quenching with CneOH, and finally oxidation with camphor sulfonyl oxaziridine, similarly to Example 3 in neat acetonitrile. Again, after removing low MW reagent debris by OSN, the temporary 5′-Dmtr protecting group was unblocked with 2.5% TFA (thermostatted 30 degrees C.), the detritylation quenched with excess 3-picoline, and diafiltration continued until no building block debris 5 remained. This cycle was repeated, injecting reagents into the synthesiser in the desired order to build up sequence 14, and removing excess reagents and debris by OSN.

As the length of the oligo on the PEG-star support increased, the permeate flux dropped slowly. This effect was thought to be caused by the increasing viscosity of the PEG-star solution. Eventually, at 11-mer-star (polymer-first compound ratio of 2.36) the viscosity of the retentate became so high that the round cells were replaced by a flat sheet stack cell (3×170 cm²); the increase in membrane area relative to the reactor volume provided a practical permeate flux, allowing building block debris 5 to be removed in reasonable time, with concurrent drying by permeation of adventitious water. The chain extension cycles continued, and the viscosity of the final 91 g/L 21-mer-star solution in MeCN was 530 cP at 30 degrees C. The 21-mer-star (polymer-first compound ratio of 1.17) was washed from the synthesiser and deprotected by ammonolysis to give 21-mer 14 with 50.2% UV-purity, FIG. 6.

While specific embodiments of the invention have been described herein for the purpose of reference and illustration, various modifications will be apparent to a person skilled in the art without departing from the scope of the invention as defined by the appended claims.

### REFERENCES

1. Lutz J. F., Ouchi M., Liu D. R., “Sequence Controlled Polymers” Science 2013, 341, 1238149-1
2. Hartmann L., Borner H. G., “Precision Polymers: Monodisperse, Monomer-Sequence-Defined Segments to Target Future Demands of Polymers in Medicine” Adv. Mater. 2009, 21, p 3423
3. U.S. Pat. No. 9,914,746
4. Lonnberg, Synthesis of oligonucleotides on a soluble support, 2017
5. Matsuno, Y.: Shoji, T.: Kim, S.; Chiba, K. Synthetic Method for Oligonucleotide Block by Using Alkyl-Chain-Soluble Support. Org. Lett. 2016, 18 (4), 800-803
6. Bonora, HELP (High Efficiency Liquid Phase) new oligonucleotide synthesis on soluble polymeric support, Nucleic Acids Research, 1990, Vol. 18, 3155; Large scale, liquid phase synthesis of oligonucleotides by the phosphoramidite approach, Nucleic Acids Research, 1993, Vol. 21, 1213-1217
7. Walther, Scalable One-Pot-Liquid-Phase Oligonucleotide Synthesis for Model Network Hydrogels, J. Am. Chem. Soc. 2020, 142, 16610-16621
8. Biochemistry. 2018 Mar. 27; 57 (12): 1821-1832. doi:10.1021/acs.biochem.7b00937
9. Walther, Scalable One-Pot-Liquid-Phase Oligonucleotide Synthesis for Model Network Hydrogels, J. Am. Chem. Soc. 2020, 142, 16610-16621
10. U.S. Pat. No. 8,664,357
11. U.S. Pat. No. 9,127,123
12. U.S. Pat. No. 10,239,996
13. EP3347402
14. P. R. J. Gaffney, J. F. Kim, I. B. Valtcheva, G. D. Williams. M. S. Anson, A. M. Buswell, A. G. Livingston, Liquid-Phase Synthesis of 2′-Methyl-RNA on a Homostar Support through Organic-Solvent Nanofiltration, Chem. Eur. J., 2015, 21, 9535-9543
15. J. F. Kim, P. R. J. Gaffney, I. B. Valtcheva, G. Williams, A. M. Buswell, M. S. Anson, A. G. Livingston, Organic Solvent Nanofiltration (OSN): A New Technology Platform for Liquid-Phase Oligonucleotide Synthesis (LPOS), Org. Process Res. Dev. 2016, 20, 1439-1452
16. So Su, Peeva L. G., Tate E. W., Leatherbarrow R. J., Livingston A. G. “Organic Solvent Nanofiltration A New Paradigm in Peptide Synthesis” Org. Process. Res. & Dev. 14 (2010) pp. 1313-132
17. Yeo J. Peeva L, Chung S, Gaffney P, Kim D, Luciani C. Tsukanov S. Siebert K, Kopach M, Albericio F. Livingston A “Liquid Phase Peptide Synthesis by One Pot Nanostar Sieving (PEPSTAR)”; Angew. Chem. Int. Ed. 2021, 60, pp 7786-7795
18. PEG and derivatives of PEG have also been synthesised using membrane separation, as described by Dong R., Liu R., Gaffney P. R. J., Schaepertoens M., Marchetti P., Williams C. M., Chen R. and Livingston A. G. “Sequence-defined multifunctional polyethers via liquid-phase synthesis with molecular sieving” Nature Chemistry (2019) 11 pp. 136-145
19. WO 2016/188835 A1
20. Peacock H. et al. J. Am. Chem. Soc. (2011). 133, 9200
21. V Kungurtsev. J Laakkonen. AG Molina. P Virta-Eur. J. Org. Chem. 2013, 6687-6693
22. Baran et al. Unlocking P(V): Reagents for chiral phosphorothioate synthesis, Science, Vol 361, 1234-1238. DOI: 10.1126/science.aau3369

1. A solution-phase process for the preparation of a first compound being a defined monomer sequence polymer, the process comprising the steps of:
a) growing the first compound by performing one or more sequential coupling reactions, and
b) performing membrane filtration to isolate the growing first compound;
wherein during steps a) and b), a plurality of growing first compounds are each attached at one end to a soluble synthesis support comprising:
a central hub, and
one or more solubility-enhancing polymers, each attached to the central hub;
wherein the total molecular weight of the one or more solubility-enhancing polymers present within each molecule of soluble synthesis support is ≥9000 Da, and
the ratio of the total molecular weight of the one or more solubility-enhancing polymers to the total molecular weight of the plurality of first compounds during steps a) and b) is ≥0.7.

2. The solution-phase process of claim 1, wherein the total molecular weight of the one or more solubility-enhancing polymers present within each molecule of soluble synthesis support is ≥9500 Da.

3. The solution-phase process of claim 1, wherein the total molecular weight of the one or more solubility-enhancing polymers present within each molecule of soluble synthesis support is ≥20,000 Da.

4. The solution-phase process of claim 1, wherein the ratio of the total molecular weight of the one or more solubility-enhancing polymers to the total molecular weight of the plurality of first compounds is ≥0.9 and ≤2.4.

5. The solution-phase process of claim 1, wherein the one or more solubility-enhancing polymers are selected from the group consisting of poly(alkylene glycols) (e.g. poly(ethylene glycol), polyester (e.g. poly(lactide co glycolide) and polysiloxanes (e.g. polydimethylsiloxane).

6. The solution-phase process of claim 1, wherein the one or more solubility-enhancing polymers is poly(ethylene glycol).

7. The solution-phase process of claim 1, wherein the one or more solubility-enhancing polymers is a plurality of solubility-enhancing polymers.

8. The solution-phase process of claim 1, wherein the one or more solubility-enhancing polymers is 3-4 solubility-enhancing polymers.

9. The solution-phase process of claim 7, wherein the solubility-enhancing polymers each have a molecular weight of ≥2000 Da.

10. The solution-phase process of claim 1, wherein the one or more solubility-enhancing polymers is 4 poly(ethylene glycol) polymers, each poly(ethylene glycol) polymer having a molecular weight of 2300-2800 Da.

11. The solution-phase process of claim 1, wherein the one or more solubility-enhancing polymers is 4 poly(ethylene glycol) polymers, each poly(ethylene glycol) polymer having a molecular weight of 4000-6000 Da.

12. The solution-phase process of claim 1, wherein the one or more solubility-enhancing polymers is 4 poly(ethylene glycol) polymers, each poly(ethylene glycol) polymer having a molecular weight of 8000-12,000 Da.

13. The solution-phase process of claim 1, wherein the plurality of first compounds is 3-4 molecules of the first compound.

14. The solution-phase process of claim 1, wherein the number of solubility-enhancing polymers is equal to the number of first compounds.

15. The solution-phase process of claim 1, wherein step a) comprises performing three or more sequential coupling reactions.

16. The solution-phase process of claim 1, wherein step b) comprises performing membrane filtration as part of each of the sequential coupling reactions forming step a).

17. The solution phase process of claim 1, wherein for each coupling reaction, membrane filtration is performed to (i) separate the supported growing first compound from a reaction by-product formed as part of a coupling reaction, and/or (ii) separate the supported growing first compound from an excess reagent used as part of a coupling reaction.

18. The solution-phase process of claim 7, wherein each one of the plurality of growing first compounds is attached at one end to a solubility-enhancing polymer.

19. The solution-phase process of claim 1, wherein the first compound is an oligonucleotide or a peptide.

20. The solution-phase process of claim 1, wherein the first compound is an oligonucleotide.

21. The solution-phase process of claim 1, wherein each first compound has a molecular weight of ≥1000 Da or ≥5000 Da.

22. The solution-phase process of claim 1, wherein membrane filtration is membrane diafiltration.

23. The solution-phase process of claim 1, wherein membrane filtration is performed using a crosslinked poly(benzimidazole) membrane.

24. The solution-phase process of claim 1, wherein steps a) and b) are conducted in the same solvent.

25. The solution-phase process of claim 1, further comprising the step:
c) cleaving the first compound, once fully grown, from the soluble synthesis support.
